SEQUENCE LISTING (4 sequences)

SEQ ID NO: 1 - DNA, 57 bp, Artificial Sequence
  Synthetic construct
  misc_feature (1)..(1): n is a, c, g, or t
  misc_feature (26)..(34): n is a, c, g, or t
nbaaaaaaaaaaaaaaaaaaaaaabnnnnnnnnngtgacctgtcccagcgtctccac

SEQ ID NO: 2 - DNA, 45 bp, Artificial Sequence
  Synthetic construct
caagcagaagacggcatacgagatccagagcctctctatgggcag

SEQ ID NO: 3 - DNA, 19 bp, Artificial Sequence
  Synthetic construct
ctgggctacactgagcacc

SEQ ID NO: 4 - DNA, 21 bp, Artificial Sequence
  Synthetic construct
gagccgcaccttgtcatgtac

(19) United States
(12) Patent Application Publication
(10) Pub. No.: US 2023/0151355 A1
(43) Pub. Date: May 18, 2023

(54) Methods for Single Cell Intracellular Capture and its Applications

(71) Applicant: Universal Sequencing Technology Corporation, Canton, MA (US)

(72) Inventors: Zhoutao Chen, Carlsbad, CA (US); Devin Porter, Carlsbad, CA (US)

(21) Appl. No.: 17/438,217

(22) PCT Filed: Mar. 12, 2020
(86) PCT No.: PCT/US2020/022455

§ 371 Date: Sep. 10, 2021

Related U.S. Application Data
(60) Provisional application No. 62/817,106, filed on Mar. 12, 2019.
(60) Provisional application No. 62/858,270, filed on Jun. 6, 2019.

(51) Int. Cl. C12N 15/10; C12Q 1/6806

(52) U.S. Cl. CPC C12N 15/1065; C12Q 1/6806; C12Q 1/6813

(57) ABSTRACT

The present disclosure provides methods for high throughput barcoding nucleic acids and/or protein inside the cells. The in-cell single cell capture method uses an individual cell itself as a compartment and delivers a plurality of unique identifiers, e.g. barcodes into the cell and captures the nucleic acid and/or protein targets within the cell directly. It significantly simplifies single cell analysis experimental setup and eliminates the need of external compartment generation. It provides a high throughput single cell expression profiling and cellular protein quantitation method. Targeted sequencing with in-cell capture will be able to significantly increase sensitivity and specificity for low frequent mutation detection, such as, somatic mutation in very early stage of cancer and truly enables early cancer detection.

### CROSS REFERENCE TO RELATED APPLICATIONS

This patent application is a U.S. National Phase application under 35 U.S.C. 371 of International Application No. PCT/US2020/022455 filed Mar. 12, 2020, which claims the benefit of and priority to U.S. Provisional Patent Application No. 62/817,106, filed on Mar. 12, 2019 and 62/858,270, filed on Jun. 6, 2019, the entire disclosure of each of which is hereby incorporated herein by reference. All publications, patents and other documents mentioned herein are incorporated by reference in their entirety.

### REFERENCE TO SEQUENCE LISTING

This specification includes a sequence listing submitted herewith, which includes the file entitled “168460-010901_ST25.txt” created on May 18, 2022, having a size of 1,340 bytes, the content of which is incorporated herein by reference in its entirety.

### FIELD

The present invention relates in general methods for single cell assay and sequencing. In particular, the methods provided herein relates in preparation of nuclei acid and/or protein capture from individual cells in a massively parallel scale and its applications on cell identification, gene expression profiling, genotyping, tumor cell detection and protein quantitation.

### BACKGROUND

A significant number of genomes from a variety of different species have been sequenced in the past decade. There are many more tissues and cell samples which have been sequenced for their genomic characteristics and transcriptome profiling. Cells in the same tissue are often considered to be functional units with the same state. In most cases, the sequenced nucleic acid samples are extracted from hundreds to millions of cells which are mixed together. This kind of bulk sequencing of thousands of cells analyzes overall response and steady state of a cell population, which averages out individual cell difference, and may not be able to precisely interpret the growth and development mechanism of an organism. Recent ability to study individual cells opens a new window to understand individual differences among cells (Janiszewska et al, 2015). The interaction of cells with internal and external factors in the process of proliferation, differentiation and metabolism creates many differences between cells. The composition and content of intracellular substances vary greatly even with homologous cells. Recent advances on technologies to capture single cells efficiently and accurately enable researchers to detect the subtle changes between individual cells (Spitzer and Nolan, 2016 and Zeisel et al, 2015). Single cell nucleic acid sequencing has shed light on multiple biological questions, such as, detecting new cancer cell types (Grün et al, 2015), identifying gene regulatory mechanisms (Datlinger et al, 2017), studying the dynamics of developmental processes (Li et al, 2017), and revealing the landscape of immune cells in cancer (Zheng et al, 2017). High-throughput single-cell sequencing not only analyzes the genetic heterogeneity of cells of the same phenotype, but also enables to acquire genetic information from those normally hard to culture cells.

The two popular methods for single-cell sequencing are plate-based protocols and microdroplet-based methods. Plate-based protocols like SMART-Seq2 (Picelli et al, 2013; Picelli et al, 2014; Tang et al, 2009) have higher sensitivity in gene detection but high cost of constructing sequencing library for individual cells. Correspondingly, microdroplet-based methods like Drop-seq (Klein et al, 2015 and Macosko et al, 2015), 10× Genomics Chromium and Biorad ddSEQ are more efficient in sequencing by building barcoded libraries for massive cells to analyze large number of cells in parallel with relatively low cost. These methods generally isolate a single cell and a plurality of unique barcode in the same droplet to construct a barcoded library per cell basis. This type of protocol still requires separating individual cells into different compartments with different identifiers, e.g. barcodes, and usually relies on a droplet generator to create droplets as the compartments.

This invention provides an in-cell single cell nucleic acid capture method, which is an intracellular nucleic acid barcoding reaction and uses an individual cell itself as a compartment and delivers a plurality of unique identifiers, e.g. barcodes into the cell and captures the genetic information within the cell directly without additional compartmentation. It significantly simplifies single cell experimental setup and eliminates the need of external compartment generation. Targeted sequencing with in-cell capture will be able to significantly increase sensitivity and specificity for very low frequency mutation detection, such as, identification of somatic mutation in very early stage of cancer development which is required for early cancer detection.

### SUMMARY

In one aspect, described herein are methods of barcoding intracellular nucleic acid without compartmentation. The methods include providing a plurality of clonal barcode templates, a plurality of cells and a reverse transcriptase. Transfect clonal barcode templates into the cells without compartmentation, wherein barcode template hybridizes to nucleic acids inside said cell. Transport the reverse transcriptase into the cells before transfecting clonal barcode templates into the cells or at the same time when transfecting clonal barcode templates into the cells or after transfecting clonal barcode templates into the cells. Synthesize complementary DNA using barcode template as primer inside the cells.

In one aspect, described herein are methods of barcoding intracellular nucleic acid without compartmentation. The methods include providing a plurality of clonal barcode templates, a plurality of cells and a reverse transcriptase. Transfect clonal barcode templates into the cells without compartmentation, wherein barcode template hybridizes to nucleic acids inside said cell. Transport the reverse transcriptase into the cells before transfecting clonal barcode templates into the cells or at the same time when transfecting clonal barcode templates into the cells or after transfecting clonal barcode templates into the cells. Synthesize complementary DNA using barcode template as primer inside the cells. Add transpososomes into the cells and perform strand transfer reaction or tagmentation reaction on the RNA/cDNA hybrid inside the cells.

In one aspect, described herein are methods of barcoding intracellular nucleic acid without compartmentation. The methods include providing a plurality of clonal barcode templates on microparticles, a plurality of cells and a reverse transcriptase. Transfect clonal barcoded microparticles into the cells without compartmentation, wherein barcode template on the microparticle hybridizes to nucleic acids inside said cell. Transport the reverse transcriptase into the cells before transfecting clonal barcoded microparticles into the cells or at the same time when transfecting clonal barcoded microparticles into the cells or after transfecting clonal barcoded microparticles into the cells. Synthesize complementary DNA using barcode template as primer inside the cells.

In one aspect, described herein are methods of barcoding intracellular nucleic acid without compartmentation. The methods include providing a plurality of clonal barcode templates on microparticles, a plurality of cells and a reverse transcriptase. Transfect clonal barcoded microparticles into the cells without compartmentation, wherein barcode template on the microparticle hybridizes to nucleic acids inside said cell. Transport the reverse transcriptase into the cells before transfecting clonal barcoded microparticles into the cells or at the same time when transfecting clonal barcoded microparticles into the cells or after transfecting clonal barcoded microparticles into the cells. Synthesize complementary DNA using barcode template as primer inside the cells. Add transpososomes into the cells and perform strand transfer reaction or tagmentation reaction on the RNA/cDNA hybrid inside the cells.

In one aspect, described herein are methods of barcoding intracellular nucleic acid without compartmentation. The methods include providing a plurality of clonal barcode templates and a plurality of cells. Transfect clonal barcode templates into the cells without compartmentation, wherein barcode template hybridizes to nucleic acids inside the cell. The methods further include lysing the transfected cells without separating the barcode template from hybridized nucleic acid, providing a reverse transcriptase and synthesizing complementary DNA using barcode template as primer.

In one aspect, described herein are methods of barcoding intracellular nucleic acid without compartmentation. The methods include providing a plurality of clonal barcode templates and a plurality of cells. Transfect clonal barcode templates into the cells without compartmentation, wherein barcode template hybridizes to nucleic acids inside the cell. The methods further include lysing the transfected cells without separating the barcode template from hybridized nucleic acid, providing a reverse transcriptase and synthesizing complementary DNA using barcode template as primer.

Add transpososomes into the reaction and perform strand transfer reaction or tagmentation reaction on the RNA/cDNA hybrid directly.

In one aspect, described herein are methods of using intracellular barcoded nucleic acid for second strand cDNA synthesis using template switch method or using general second strand cDNA synthesis method, such as with RNaseH/DNA polymerase/DNA Ligase combination.

In one aspect, described herein are methods of using intracellular barcoded nucleic acid to prepare sequencing libraries for single cell expression profiling, single cell targeted sequencing and immune repertoire analysis.

In one aspect, described herein are methods of detecting early stage cancer. The methods including providing test sample as separate cells, barcoding intracellular nucleic acid to generate cellular barcode-tagged complementary DNA, generating sequencing library covering regions containing one or more tumorigenic variants and cellular barcode tag using the complementary DNA, grouping sequencing reads based on their cellular barcode sequences and determining the presence of tumorigenic variants on a per cell basis and counting tumor cell number and determining tumor cell percentage in the test sample.

In one aspect, described herein are methods of barcoding intracellular protein without compartmentation. The methods include providing a plurality of protein capture moieties with a first barcode template and a plurality of cells, the protein capture moieties bind to specific targeted endogenous protein inside the cells; providing a plurality of a second clonal barcode templates; transfect the second clonal barcode templates into the cells without compartmentation, wherein the second barcode template hybridize to the first barcode template on the capture moiety which captures targeted endogenous protein inside the cells. Release the barcoded templates with protein captured from the cells and sequence the barcode templates to determine captured protein level on a per cell basis.

In one aspect, described herein are methods for cell-specific intracellular nucleic acid barcoding without compartmentation. The methods include contacting a plurality of cells with a plurality of clonal barcode templates, wherein each clone comprises a cell-specific anchor; anchoring clones of barcode templates to a specific type of cells by the cell-specific anchor; transfecting the clonal barcode templates into the type of cells without compartmentation, wherein the barcode template hybridizes to nucleic acids inside the cell; analyze gene expression or genotype of the anchored cell at per cell basis based on the barcode information.

In one aspect, described herein are methods of barcoding intracellular nucleic acid without compartmentation for targeted applications. The methods include providing clonal barcode templates with a first set of target specific primers which are used for in-cell capture of specific nucleic acid target(s). Transfect clonal barcode templates and the first set of target specific primers into the cells without compartmentation. Perform reverse transcription reaction either inside the cells or after cell lysis, collect clonal barcoded templates with targeted first strand cDNA and further prime the first strand cDNA with a second set of target specific primers to generate double stranded DNA for downstream applications, including tagmentation, amplification or sequencing library generation.

In one aspect, described herein are methods of barcoding intracellular nucleic acid without compartmentation for targeted applications. The methods include providing clonal barcode templates with a set of target specific primers which are used for in-cell capture of specific nucleic acid target(s). Transfect clonal barcode templates and the target specific primers into the cells without compartmentation. Perform reverse transcription reaction either inside the cells or after cell lysis, collect clonal barcoded templates with targeted first strand cDNA, perform strand transfer reaction or tagmentation reaction with transpososomes on the RNA/cDNA hybrid double-stranded molecule for downstream applications including amplification or sequencing library generation.

In one aspect, described herein are methods of in-cell barcoding and capture of DNA from nuclei or mitochondria. The methods include a fixation step before or after transfection of clonal barcoded templates into the cells.

In one aspect, described herein are methods of barcoding intracellular nucleic acid without compartmentation. Adjust the ratio of clonal barcode templates to cells for different applications. In general, one type of clonal barcode templates in a cell is preferred. Greater than one type of clonal barcode templates in a cell is used for genetic variation detection and immune repertoire analysis. This condition can also be used for quantitative analysis such as gene expression profiling when cellular origin of different type of clonal barcode templates can be identified with additional computation approach.

In one aspect, a method for intracellular barcoding targets without compartmentation is disclosed comprising: providing a barcode template, providing a cell, transfecting the barcode template into the cell; and wherein when the cell is in the context of a plurality of other cells, the cell is not separated by a partition from the other cells, and wherein the barcode template captures an intracellular target inside the cell, generating a nucleic acid sequence derived from the captured intracellular target inside the cell or from the captured intracellular target after the cell is lysed, and wherein the nucleic acid sequence is attached to a barcode sequence or a complementary barcode sequence from the barcode template, and identifying the nucleic acid sequence and/or its complementary sequence as being derived from the cell based on the presence of the barcode sequence attached; and wherein another nucleic acid sequence with the same barcode sequence correlates with the same cell. In another aspect, the barcode template is amongst a plurality of barcode templates and they are immobilized on a microparticle; wherein the microparticle size ranges from about 100 nm to about 100 μm; and wherein the plurality of barcode templates are clonal with respect to each other. The microparticle size ranges from 1 μm to 20 μm. In an aspect, the microparticle is magnetic or degradable.

In one aspect, the microparticle is among a plurality of microparticles; wherein a population within the plurality of microparticles are non-barcoded and another population within the plurality of microparticles comprise barcode templates; wherein the two populations are mixed; and wherein the barcode templates in the population of microparticles with barcode templates are clonal with respect to each microparticle.

In one aspect, the barcode template is non-immobilized. The non-immobilized barcode template is encapsulated in a liposome or a droplet; wherein within each liposome or each droplet, the barcode template is clonal with respect to another barcode template in the liposome or in the droplet.

In an aspect, a clone of the barcode template comprises about 10 or more copies of the barcode template. In another aspect, the clone of the barcode template comprises about 10,000 or more copies. In another aspect, the clone of the barcode template comprises about 10,000,000 or more copies.

In another aspect, on a microparticle or within a liposome or a droplet, the barcode template is clonal with respect to another barcode template in a population of barcode templates; and wherein more than one population of barcode templates exists on the microparticle or within the liposome or the droplet. On a microparticle or within a liposome or a droplet, more than one population of barcode templates exists, wherein the first population of barcode template is clonal with respect to the first population of another barcode template in a plurality of barcode templates; and the second population of barcode template is the same with respect to the second population of another barcode template in a plurality of barcode templates.

In an aspect, the barcode template is among a plurality of barcode templates and the cell is among a plurality of cells; wherein a ratio of the barcode templates to the cells is such that when the barcode templates are transfected into the cells, less than about 30% of the transfected cells comprise more than one clonal population of barcode templates, and more than about 70% of the transfected cells comprise one or less clonal population of barcode templates. In another aspect, less than about 20% of the transfected cells comprise more than one clonal population of barcode templates, and more than about 80% of the transfected cells comprise one or less clonal population of barcode templates. In another aspect, less than about 10% of the transfected cells comprise more than one clonal population of barcode templates, and more than about 90% of the transfected cells comprise one or less clonal population of barcode templates.

In an aspect, within the cell, more than one population of a plurality of barcode templates is in the cell; and wherein the barcode templates within each population of barcode templates are clonal.

In an aspect, the barcode template compromising a barcode sequence, and at least one adaptor capable of priming, hybridization, amplification, strand transfer, or a combination thereof. In an aspect, one clone of the barcode template comprising an UMI sequence. In another aspect, the adaptor is selected from the group consisting of a ploy-T sequence, a target specific sequence, a pool of different target specific sequence, a random degenerate sequence, and a combination thereof.

In an aspect, the intracellular target is selected from the group consisting of a RNA, a DNA, an oligonucleotide, an oligonucleotide-labeled protein, an oligonucleotide-labeled chemical compound, and a combination thereof. In another aspect, the intracellular target enters the cell as an extracellular component through a specific recognition of the cell.

In an aspect, the cell is selected from the group consisting of a cultured cell, a blood cell, a tissue, a tissue section, a biopsy sample, a nucleus derived from the cell, and a combination thereof.

In an aspect, the cell is fixed with a fixative selected from the group consisting an alcohol, a Hepes-glutamic acid buffer-mediated organic solvent protection effect (HOPE) fixative, and a combination of thereof.

In an aspect, the transfection is facilitated by a magnetic force or by centrifugation.

In an aspect, the target capture is mediated by hybridization, ligation, strand transfer, directly or indirectly, or a combination thereof.

In an aspect, the generated nucleic acid sequence derived from the captured intracellular targets is by reverse transcription, primer extension, ligation, amplification, tagmentation, or a combination thereof.

In an aspect, wherein the intracellular barcoded target is used for single cell expression profiling, single cell targeted sequencing, immune repertoire analysis and/or single cell protein analysis.

In an aspect, a method for intracellular protein barcoding without compartmentation is disclosed comprising providing a barcode template, providing a protein capture moiety that comprises a capture site caught by the barcode template and a capture site to a target protein, providing a cell, transfecting the protein capture moiety into the cell wherein the cell is not compartmentalize with respect to another cell, and wherein the protein capture moiety is configured to bind to the target protein inside the cell when the target protein is present, transfecting the barcode template into the cell wherein the cell is not compartmentalize with respect to another cell, and wherein the barcode template binds to the protein capture moiety inside the cell when the target protein is present, generating one or more nucleic acid sequences derived from the barcode template and the bound protein capture moiety inside the cell or after cell is lysed, wherein the one or more nucleic acid sequences have attached to the same barcode sequence or complementary barcode sequence from the barcode template in the cell, and sequencing the nucleic acid sequence to determine the target protein level on a per cell basis based on the presence of a common barcode sequence and capture moiety derived sequences. In another aspect, the barcode template is amongst a plurality of barcode templates; the plurality of barcode templates are clonal with respect to each other.

In an aspect, a method for cell-specific intracellular nucleic acid barcoding without compartmentation is disclosed comprising (a) providing a barcode template, wherein the barcode template comprises a cell-specific anchor, (b) providing a cell, contacting the clonal barcode template of (a) and the cell of (b), and anchoring a clone of the barcode template to a specific cell type by the cell-specific anchor, transfecting the clonal barcode template into the cell wherein the cell is not compartmentalize with respect to another cell, and wherein the barcode template hybridizes to a nucleic acid target inside the cell, generating one or more nucleic acid sequences derived from the barcode template and the hybridized nucleic acid target inside the cell or after cell is lysed, wherein the one or more nucleic acid sequences are attached to the same barcode sequence from the barcode template in the cell, and sequencing the nucleic acid sequence to characterize the nucleic acid target in the specific cells on a per cell basis based on the presence of a common barcode sequence. In another aspect, the barcode template is amongst a plurality of barcode templates; the plurality of barcode templates are clonal with respect to each other.

In an aspect, a method for early disease detection is disclosed comprising providing a test sample comprising (a) a cell or cellular component, (b) barcoding a nucleic acid inside the cell or cellular component to generate a cellular barcode-tagged nucleic acid sequence; generating a sequencing library wherein the sequencing library comprises a region containing one or more disease-causative variants and the cellular barcode tag using the barcode-tagged nucleic acid sequences in (b), grouping sequencing read based on their cellular barcode sequences and determining the presence of disease-causative variants on a per cell basis, and counting the number of the cells containing disease-causative variants among the test sample. In another aspect, the barcode template is amongst a plurality of barcode templates; the plurality of barcode templates are clonal with respect to each other.

### BRIEF DESCRIPTION OF THE DRAWINGS

FIG. 1 illustrates a polymerization method to generate microparticles with immobilized poly-T tailed oligos on the surface.

FIG. 2 illustrates a polymerization method to generate microparticles with immobilized poly-T tailed oligos, which also contain a unique molecular identifier (UMI) sequence. A) a structure of immobilized single stranded barcode template comprising a UMI and poly-T tail at the 3′ end; B) hybridizing a UMI and poly-T oligo to a clonally barcoded microparticle before using polymerization method to generate a microparticle shown in A.

FIG. 3 illustrates a ligation-based method to generate microparticles with immobilized poly-T tailed oligos on the surface.

FIG. 4 illustrates a method of capturing nucleic acid inside a single cell directly using clonal barcode oligo coated microparticles and followed by an extracellular reverse transcription reaction to generate a barcoded microparticle with complementary DNA synthesized from captured nucleic acid targets.

FIG. 5 illustrates a method of capturing nucleic acid inside a single cell directly using clonal barcode oligo coated microparticles and followed by an intracellular reverse transcription reaction to generate a barcoded microparticle with complementary DNA synthesized from captured nucleic acid targets.

FIG. 6 illustrates methods to improve transfection efficiency of oligo coated microparticle into cells. (A) for suspended cells (including cells from homogenized tissue), make the cells settle down to the bottom of the container loosely by centrifugation before addition of oligo coated microparticles; (B) for adhesive cells; transfect the oligo coated microparticle into the cells with the help of centrifugation force and/or magnetic force.

FIG. 7 illustrates a method of capturing nucleic acid inside a single cell directly using non-immobilized clonal barcode oligos.

FIG. 8 illustrates methods of generating targeted capture library for a single cell based targeted gene expression analysis and/or genotyping analysis using in cell captured nucleic acid for one target or multiple targets.

FIG. 9 illustrates that in cell targeted sequencing can significantly improve detection power of somatic mutation with the combined ability for cell identification and unique molecule identification.

FIG. 10 illustrates a single cell transcriptome application using incell captured nucleic acids with template switch reaction.

FIG. 11 illustrates a single cell transcriptome application using in-cell captured mRNA with intracellular tagmentation on DNA/RNA hybrid directly.

FIG. 12 are pictures of HCT116 cells transfected with TELL beads.

FIG. 13 is a picture of PCR products run on a 2% e-gel EX. Products in Lane 1 and Lane 5 were results from successful In-Cell capture of GAPDH mRNA to poly-T extended TELL beads and in situ reverse transcription to generate first strand cDNA. Lane 3 and Lane 5 were positive controls using extracted mRNA instead of cells as reaction input. Lane 2, 4, 6 and 8 were negative controls without reverse transcriptase in the reaction.

### DETAILED DESCRIPTION

Individual cells are different. Even isogenic populations of cells showed substantial cell-to-cell heterogeneities than we previously thought. By using averaged molecular or phenotypic measurements of a cell population to represent an individual cell behavior, conclusions could be biased by the expression profiles of a majority group of cells or over-expressed outliers; and we will not have the sensitivity to identify all unique patterns from an individual cell which could be distinctive functional behaviors for a cell at a given place and time. Studying single cells offer a new window to understand individual differences among cells. Large-scale surveys of single-cell gene expression have the potential to reveal rare cell populations and lineage relationships but require efficient methods for cell capture and mRNA sequencing (Kawaguchi A et al, 2008; Shalek A K et al, 2013; Shapiro E et al, 2013; Treutlein B et al, 2014). In addition, early tumor detection has been significantly restrained by limited ability to detect very low frequent somatic mutation currently due to presence of high background wild type signal from normal cells or tissue. However, with improved ability to identify every single cell, we will be able to separate the mutant tumor cells from wild type cells by genotyping at single cell level. This will remove the wild type background signal generated from normal cells completely and make somatic mutation detection as easy as germline mutation detection.

Two commonly used methods for single-cell sequencing are plate-based protocols and microdroplet-based methods. Plate-based protocols have higher sensitivity in gene detection, but costly library construction for each cell, very time consuming, and hard to scale up the process for thousands of cells. Microdroplet-based methods are more efficient in sequencing by building one barcoded library for massive cells to analyze large number of cells in parallel with relatively low cost. It requires to separate each cell into a compartment with a plurality of unique barcode for sequencing library generation, which needs a specially designed microfluidic device usually.

This invention provides an in-cell single cell capture method, which directly captures nucleic acid within a cell without any additional compartment to sequester each cell. Capturing mRNAs inside of a cell, instead of outside, is a more efficient way of capturing mRNA molecules and should allow for near complete mRNA capture. This will overcome the low mRNA capture efficiencies and high drop-out rate of conventional single cell capture method (Bagnoli et al 2018). This intracellular nucleic acid capture reaction dramatically simplifies the sample preparation workflow for single cell expression analysis, single cell genotyping and sequencing analysis, and will offer a cost-effective solution for single cell-based studies.

The in-cell single cell capture method is built on decades of knowledge on in situ hybridization, live cell imaging study and DNA transfection technologies.

Localizing mRNA with labeled linear oligonucleotide (ODN) probes in a cell have been demonstrated long time ago via in situ hybridization (Bassell G J et al, 1994) in which cells are fixed and permeabilized to increase the probe delivery efficiency.

Furthermore, live cell imaging technologies developed in the past decade have showed that oligonucleotide probe can bind to mRNA within a live cell (Kam Y et al, 2012; Okabe K et al, 2011; Rodrigo J P et al, 2005). For both in situ hybridization and live cell imaging, oligonucleotide probes need to be delivered into targeted cells. In general, transfection is the process of deliberately introducing naked or purified nucleic acids into eukaryotic cells. There are various methods of introducing foreign DNA into a eukaryotic cell. Some rely on physical treatment (electroporation, cell squeezing, nanoparticles, magnetofection); Others rely on chemical materials or biological particles (viruses) that are used as carriers. Among the delivery mechanism based on physical treatment, there are several particle-based methods, such as gene gun, magnetofection (Hughes C et al, 2001; Krotz F et al, 2003; Scherer F et al, 2002), impalefection (McKnight T E et al, 2004) and particle bombardment (Uchida M et al, 2009), etc. Magnetofection, or magnet-assisted transfection, is a transfection method that uses magnetic force to deliver DNA into target cells. Nucleic acids are first associated with magnetic nanoparticles. Then, application of magnetic force drives the nucleic acid particle complexes towards and into the target cells, where the cargo is released. This approach has successfully demonstrated that magnetic particles associated with nucleic acid cargo can get into a cell efficiently under proper conditions.

In-cell single cell capture method is to transfect clonally barcoded templates, which are unique sequences used as a cellular identifier, into cells and hybridize the barcode templates to nucleic acid targets inside the cell directly.

The term “barcode” as used herein refers to a nucleic acid sequence that is 5 to 100 nucleotides and is used as an identifier.

The term “barcode template” as used herein refers to a nucleic acid sequence comprising a barcode and at least one adaptor. The nucleic acid sequence can be a DNA, RNA or DNA/RNA mixture.

The term “clonal barcode templates” as used herein refers to a plurality of barcode templates with the same barcode sequence. They can be delivered in various formats including in a droplet, in a liposome, on a microparticle, as a nanoball or a combination thereof.

The term “adaptor” as used herein refers to a nucleic acid sequence that can comprise one or more of following: a primer binding sequence, a barcode, a capture sequence, a unique molecular identifier (UMI) sequence, an affinity moiety, restriction site, a ligand, a transposon, or a combination thereof.

The term “microparticle” as used herein refers to a particle, a sphere or a bead or any other shape of solid material with sizes smaller than 1 mm, preferable between 0.1 μm and 100 μm.

The term “clonal” as used herein refers to a plurality of the same molecule.

The term “transfection” as used herein refers to methods that transport a nucleic acid material into a cell.

The term “capture” as used herein refers to a binding reaction from one or more of following: hybridization, ligation, affinity moiety binding, click reaction, cross-linking, antibody to antigen binding, ligand to receptor binding, or a combination thereof.

The term “in-cell” as used herein refers to inside a cell or intracellular.

The term “transposase” as used herein refers to a protein that is a component of a functional nucleic acid protein complex capable of transposition and which is mediating transposition, including but not limited to Tn, Mu, Ty, and Tc transposases. The term “transposase” also refers to integrases from retrotransposons or of retroviral origin. It also refers to wild type protein, mutant protein and fusion protein with tag, such as, GST tag, His-tag, etc. and a combination thereof.

The term “transpososome” as used herein refers to a stable nucleic acid and protein complex formed by a transposase non-covalently bound to a transposon. It can comprise multimeric units of the same or different monomeric unit.

A “strand transfer reaction” as used herein refers to a reaction between a nucleic acid and a transpososome, in which stable strand transfer complexes form.

A “tagmentation reaction” as used herein refers to fragmentation reaction where transpososomes insert into a target nucleic acid through strand transfer reaction and form strand transfer complexes, and strand transfer complexes are then broken under certain conditions, such as, protease treatment, high temperature treatment, or a protein denaturing agent, e.g. SDS solution, guanidine hydrochloride, urea, etc., or a combination thereof, so that the target nucleic acid breaks into small fragments with transposon end attached.

Prepare Clonally Barcoded Microparticle with a Capture Sequence

We developed a process to prepare clonally or semi-clonally barcoded microparticles as described in patent application WO2017/151828, which is hereby incorporated by reference in its entirety. In some embodiment, the clonal barcoded microparticles are generated by clonal amplifications. In some embodiment, the clonal barcoded microparticles are generated by direct synthesis on the microparticle surface.

In some embodiment, the clonal barcoded microparticles are generated by multiple rounds of ligation based split and pool method.

As used herein and in the appended claims, a barcode template and a solid support with clonal barcode templates or semi-clonal barcode templates immobilized thereon are also described in patent application WO2017/151828, which is hereby incorporated by reference in its entirety. In this invention, the solid support are microparticles or beads preferably.

In some embodiments, all the solid support has barcode templates attached. In some embodiments, only a fraction of solid support has barcode templates attached. The fraction of solid support with barcodes can be ranged from 1% to 100%.

In order to capture nucleic acid broadly, a random degenerate sequence, ranging from 4-mer to 20-mer can be attached to the 3′ end of the barcode template on the clonally barcoded microparticle.

In order to capture 3′ end of mRNA specifically, a poly-T tail, which contains 15 to 40 deoxythymines, needs to be added at the 3′ end of the barcode template on the clonally barcoded microparticle. In some embodiment, a V (dATP, dCTP or dGTP) or VN (dATP, dCTP, dGTP or dTTP) nucleotide is added at the 3′ end of poly-T tail to improve the mRNA capture efficiency.

In one embodiment, a poly-T sequence can be added at the 3′ and distal end of barcode template design and used for clonal amplification to generate clonal barcoded microparticle with a poly-T tail on all the barcode oligos. In another embodiment, the poly-T sequence can be incorporated to the barcode template during clonal amplification with poly-A tailed primers.

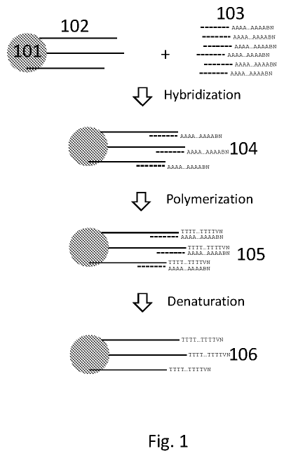

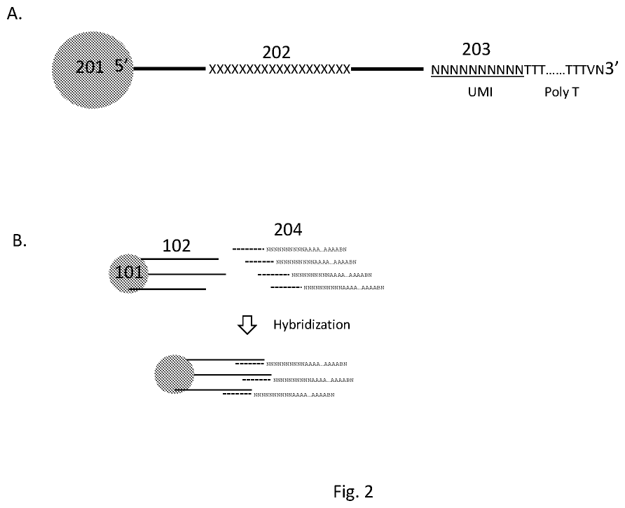

In some embodiment, poly-T tails can be added later after clonally barcoded microparticles have been prepared as described in patent application WO2017/151828. One method is illustrated in FIG. 1. Briefly, poly-A tailed oligos (103) hybridize to the single stranded (102) clonally barcoded microparticle (101). With a polymerase which can generate a blunt end double stranded DNA, a poly-T sequence will be added to each immobilized barcode template on the microparticles (105) after a fill-in reaction. The poly-A primer or strand can be removed from the microparticle under denaturation conditions. In some embodiment, a degenerate sequence (203) which can be used as unique molecular identifier of each barcode (202) template is a part of poly-A tailed primers (204). Using the same hybridization and polymerization method as FIG. 1, each barcode template can be extended with a unique random sequence (UMI) and a poly-T tail (FIG. 2A).

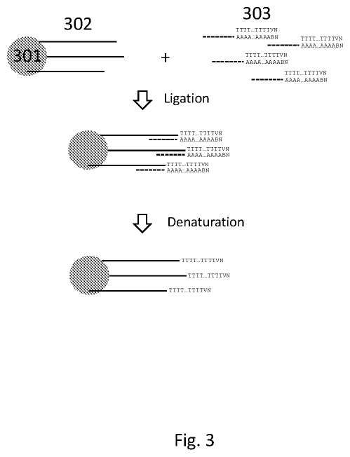

In some embodiment, a ligation-based method can be used to add poly-T tail to clonally barcoded template (FIG. 3). One advantage with this method is that any modification to the poly-T sequences, such as using phosphorothioate to protect poly-T tail against nuclease degradation, can be easily to incorporate in the ligation linker which contains the poly-T sequences (303). Both double stranded ligation and single stranded ligation can work for this purpose.

In order to capture target specific nucleic acids, a target specific primer or a pool of target specific primers can be attached to the 3′ end of clonally barcoded microparticle instead of a poly-T tail described previously using either hybridization & fill-in method as FIG. 1 or ligation method as FIG. 3.

Use Barcoded Microparticles for In-Cell Capture of Nucleic Acid

During the last decade, transfection of nucleic acids using nanomagnetic particles has been developed and shows high transfection efficiency and low toxicity. The method is often referred to as magnetofection (Hughes C et al, 2001; Krotz F et al, 2003; Scherer F et al, 2002). Magnetofection, or magnet-assisted transfection, is a transfection method that uses magnetic force to improve delivery of DNA into target cells. Nucleic acids are first associated with magnetic nanoparticles. Then, application of magnetic force drives the nucleic acid particle complexes towards and into the target cells, where the cargo is released. Magnet-assisted particle-based transfection becomes much more popular than the non-magnetic particle-based transfection methods, however, studies have indicated that there are probably no fundamental mechanistic differences between magnetofection and gene delivery with analogous non-magnetic vectors (de Bruin K et al, 2007; Huth S et al, 2004; Namgung R et al, 2010; Sauer A M et al, 2009). Polyethylenimine (PEI) is often used to pack DNA and nanoparticles together before transfection. DNA with PEI-coated nanoparticles binds to the cell surface. The PEI-DNA complexes including the nanoparticles are internalized into intracellular vesicles called endosomes by the natural uptake process of endocytosis. Escape from endosomes is essential for functional nucleic acid delivery because otherwise vectors would be degraded by the cellular breakdown machinery (Plank C et al, 1994). PEI-DNA complexes are thought to escape due to the so-called proton sponge effect (Boussif O et al, 1995).

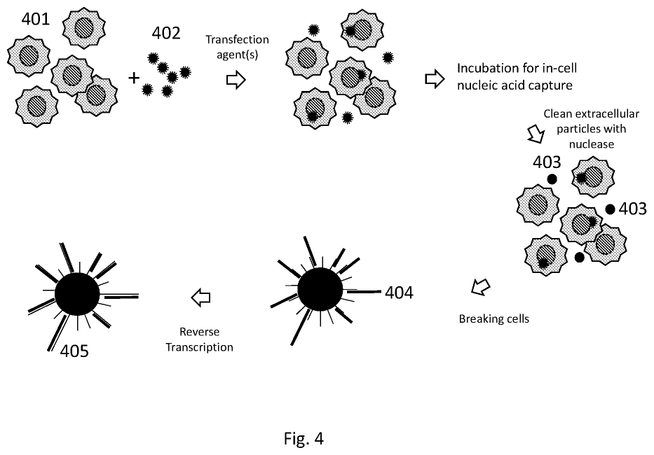

In some embodiments of the in-cell capture method provided in this invention, particle-based transfection methods are used to deliver the barcoded microparticle into the target cells (FIG. 4). Individual cells (401), such as, cells from tissue culture or lymphocytes from blood, cells from homogenized tissue, are collected in a tube or a plate. Barcoded microparticle (402) are transfected into target cells with or without magnetic force assistance. Microparticle size can be ranged from 10 nm to 50 μm, preferably 100 nm to 20 μm. In some embodiment, optimized microparticle to cell ratio will be used to reduce the probability of multiple particles entering one cell. In some embodiment, microparticles without barcode templates were mixed with clonal barcoded microparticles and act as a spacer to keep barcoded microparticle apart. In some embodiment, more than one barcoded microparticle to cell ratio will be used to increase the proportion of cells with at least one barcoded microparticle. This condition will work effectively for immune repertoire sequencing to gather paired heavy chain and light chain information of an antibody from B cells or paired alpha and beta chain information of a TCR from T cells. It will also work for detecting genetic variants and targeted sequencing applications when quantitative information at per cell level is not critical. Additional computation method can be developed to identify the cellular origin of the different barcodes based on their shared nucleic acid sequences. When barcoded microparticles enter the target cells, barcoded capture sequences on the microparticle will capture mRNA or nucleic acid target in the cells by hybridization or ligation after a period of incubation. For the barcoded microparticles which are left outside of cells, addition of single stranded DNA specific nuclease will degrade the oligos on the microparticle surface (403). The cells are broken with proteinase K, SDS, high salt treatment or combination of these. Released microparticles which are bound with captured mRNA or target nucleic acid (404) from the targeted cells will be isolated from the cell debris. cDNA of captured nucleic acid (405) can be synthesized on the barcoded microparticle by reverse transcription when incubate the isolated microparticle with a reverse transcriptase.

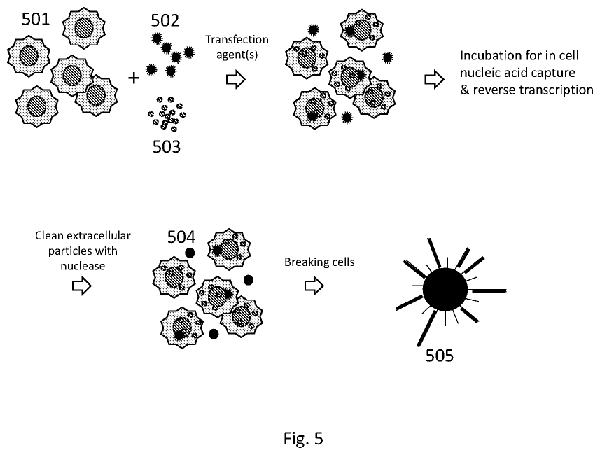

In some embodiments, reverse transcription can be performed intracellularly right after In-Cell capture reaction (FIG. 5). Reverse transcriptase (503) can be introduced with barcoded microparticles (502) at the same time or before the transfection of barcode microparticles. Cells may be treated with detergent, such as, Triton X-100 to become more permeable. Reverse transcriptase will penetrate cell membrane into cells. After barcoded capture sequences on the microparticle capture mRNA or nucleic acid target in the cells by hybridization, first strand cDNA will be generated by reverse transcription reaction intracellularly. The extracellular microparticles (504) will be cleaned to remove single stranded oligos on the surface to avoid interference in the downstream process. The cells are then lysed to release barcoded microparticles with nucleic acid captured and the first strand cDNA ready (505).

In some embodiment, transpososomes, such as Mu or Tn5 can be added and perform strand transfer reaction or tagmentation reaction on the RNA/cDNA hybrid inside the cells or outside the cells. This will simplify the downstream workflow by skipping the second strand cDNA synthesis.

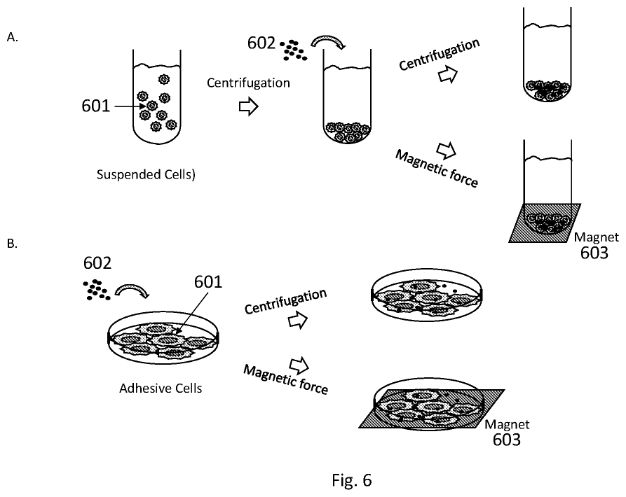

It is important to transfect barcoded microparticles into cells efficiently. Both centrifugation and magnetic force can be used to improve the transfection efficiency (FIG. 6). Tissues will be homogenized into suspended cells. Suspended cells (601) will be collected down at the bottom of a centrifugation tube loosely before or at the same time when barcoded beads (602) are added (FIG. 6A). Further centrifugation and/or applying magnetic force if barcoded microparticles are magnetic will facilitate the transfection of microparticles into cells. For adhesive cells, barcoded microparticles can be added directly on the top of cell layer (FIG. 6B). Additional centrifugation and/or magnetic force will help deliver the microparticles into the cells.

Use Non-Immobilized Clonal Barcodes for In-Cell Capture of Nucleic Acid

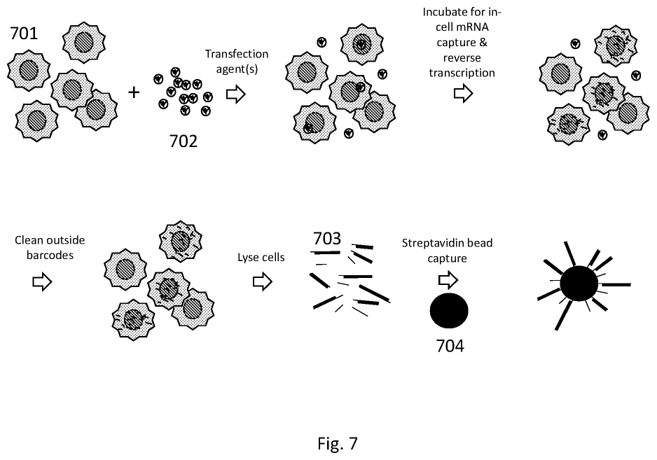

Clonally immobilized barcode templates on the surface of microparticle may have low efficiency to capture nucleic acid targets inside a cell due to limited movement. In one embodiment is to wrap clonally barcoded microparticles individually into liposomes. In one embodiment, immobilized barcode templates can be enzymatically released from the microparticles. In another embodiment, microparticles can be dissolved and release barcode templates. Such as, a hydrogel based microparticle which can be dissolved at an elevated temperature. In some embodiments, barcode templates contain a biotin label, which can be used for captured by streptavidin beads when needed. Liposomes containing released clonally barcoded templates (702) are transfected into cells of interest (FIG. 7, 701). Barcoded templates will be further released from liposome inside the cells and hybridize to its nucleic acid target(s). In some embodiment, reverse transcriptase is also delivered into the cells. First strand cDNA synthesis using capture sequence on the barcoded template as primer will attached a barcode sequence to the newly synthesized cDNA. When cells are lysed, these barcode-tagged cDNAs (703) can be captured by streptavidin beads (704) for further downstream process.

There are other ways to generate non-immobilized clonal barcode templates. In one embodiment, directly synthesized barcode templates are clonally packed into liposomes or water-in-oil emulsion droplets. In some embodiments, barcode templates are clonally amplified in water-in-oil emulsion droplets. In some embodiment, barcode templates are clonally amplified in liposomes.

Liposomes are vesicles containing lipid membranes, mimicking that of cellular membranes and exist in various sizes. Small unilamellar liposome (SUVs) range 20-100 nm in diameter, large uniamellar liposome vesicles (LUVs) range 100-1000 nm in diameter, and giant unilamellar liposome vesicles (GUVs) with a size from 1-200 um in diameter (Laouini et al 2012). In some embodiment, GUVs or LUVs are used to encapsulate a unique barcode template and primers, and at least one set of primers contains a plurality of UMI sequences, as well as other necessary reagents for oligo amplification. Clonal amplification in the liposomes will generate a plurality of barcode templates with UMI sequences attached and all barcode templates share the same barcode sequences. LUVs or SUVs can be used to encapsulate reverse transcriptase and other necessary reagents for first-strand synthesis of mRNA.

Clonally amplifiable GUVs can be prepared using Paper-Abetted amPhiphile hYdRation in aqUeous Solutions (PAPYRUS) method (Pazzi and Subramaniam 2018). In this case, the aqueous solution will barcode templates, primers and DNA polymerase in PCR buffer. The size of GUVs can be ranged from 1 μm to 10 μm in diameter. This method is easily scalable and thus millions of GUVs could be produced in a single reaction. Once GUVs are produced, 20-30 cycles of PCR amplification should be able to generate clonal amplified barcode templates. Amplification cycles should be maximized to ensure optimal amplification of the GUVs but limited to decrease the rupture of GUV liposomes. In some embodiment, SYBR green is added into the PCR amplification mix to determine the number of amplified liposomes via microscopy or FACS. FACS sorting allows for the purification of amplified GUVs by size and overall fluorescence.

Liposomes are integrated into cells via two main mechanisms, endocytosis or cell-membrane fusion (Braun et al 2016). The former requires lysosomal degradation of the endosome and may require more time for efficient delivery of barcode payload inside the cell (Parker et al 2003). In some embodiment, photo switchable lipids are added during the liposomal generation phase to bypass the lysosomal degradation of the endosome (Miranda and Lovell 2016). High power wavelengths can then be applied to cells to destabilize the liposome membrane and thus releasing the barcode payload into the cytoplasm. In some embodiment, an electrofusion method can be applied to increase the rate of cell-membrane fusion versus endocytosis (Raz-Ben Aroush et al 2015, Pereno etal 2017).

Reverse transcription can occur in many ways. In some embodiment, LUVs or SUVs encapsulating reverse transcriptase can be co-transfected into cells with GUVs containing clonally amplified barcode templates. In some embodiment, LUVs or SUVs encapsulating reverse transcriptase and GUVs containing clonally amplified barcode templates can be fused together prior to cell delivery, so that one endosome is integrated into the cell and not multiple. In some embodiment, cells can be fixed and permeabilized to allow direct intake of reverse transcriptase without liposome delivery. In some embodiment, reverse transcription of captured RNA molecules can be done after cells lysis.

In some embodiment, liposomes are used to target specific cell types by adding antibody moieties to the lipid membrane. Immunoliposomes have been created to target specific cell types for drug-delivery applications (Eloy et al 2017). These groups alter the composition of the lipid membranes to allow a thiolated antibody to covalently bind with a maleimide group on the liposomes surface (Eloy et al 2017). This immunoliposome approach applied to single-cell RNA-seq applications, provide for novel and efficient method to track T-cell states in response to immunotherapy therapeutics.

In some embodiment, liposomes can be fused with cell-derived exosomes to increase the selectiveness of cell-type delivery of the liposome's cargo. Exosomes are cell-derived, ex-membrane vesicles that are naturally secreted. They retain their membrane-protein composition which is used to communicate to other target cells (Antimisiaris et al, 2018). By fusing liposomes to cell-derived exosomes, higher rates of cell fusion is achieved (Sato et al, 2016). In some cases, the cell-derived exosomes can come from T-cells or B-cells and purified using the gold-standard ultracentrifugation method (Lu et al, 2018). Ultimately, exosome-fused liposomes will aid the delivery of clonally amplified barcodes to target-cells for nucleic acid capture.

In some embodiments, the barcode templates are designed and clonally amplified as DNA nanoballs directly without any solid support. These DNA nanoballs are transfected into the cells to capture target nucleic acids. In some embodiment, before transfection, barcoded DNA nanoball can be wrapped into a liposome or a water-in-oil emulsion droplet and nanoball structure is dissolved in the liposome or droplet first.

In some embodiment, in-cell barcoding and capture method can be modified for capture of DNA from nuclei or mitochondria specifically. Cells are treated with alcohol-based fixative or Hepes-glutamic acid buffer-mediated organic solvent protection effect (HOPE) fixative to release the DNA inside the cells for capture by barcode templates. This fixation step can be done before or after transfecting clonal barcode templates into the cells. In some embodiment, transpososomes are added and strand transfer reaction can be performed after cell fixation but before the transfection of clonal barcode templates. In some embodiment, strand transfer reaction can be performed after cell fixation and transfection of clonal barcode templates into the cells.

Applications with In-Cell Captured Nucleic Acids

In-cell captured nucleic acids from this invention can be used for a variety of downstream applications. Notably, it will be a convenient new tool for whole transcriptome analysis, targeted gene expression profiling and targeted genotyping. In-cell capture will offer an unmatchable level of sensitivity for low frequency allele detection, such as, in the case for detecting early stage of cancers. It will also be a valuable assay for immune repertoire profiling by providing paired information on the heavy and light chain of an antibody or alpha and beta chain of a TCR.

In one embodiment, in-cell captured barcoded nucleic acid after first strand cDNA synthesis will go through second strand cDNA synthesis using template switch method or with second strand cDNA synthesis kit to generate barcoded double stranded cDNA before further use.

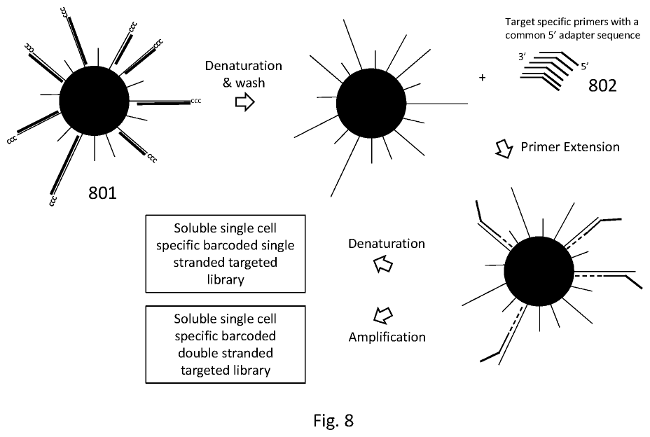

In one embodiment, barcoded microparticles with a target specific primer or a pool of target specific primers are used for in-cell capture of specific nuclei acid target(s). After reverse transcription reaction is completed either inside the cells or after cell lysis, barcoded microparticles with the first strand cDNA are collected after cell lysis (FIG. 8, 801). The original copies of nucleic acid targets are removed by denaturation and barcoded microparticles with single stranded cDNA copy can be further primed with a target specific primer or primer pool (802) to generate double stranded amplifiable templates for downstream applications, such as, PCR assay and/or library construction for sequencing.

In one embodiment, barcoded microparticles with a first set of target specific primers are used for in-cell capture of specific nucleic acid target(s). Transfect clonal barcode templates and the first set of target specific primers into the cells without compartmentation. Perform reverse transcription reaction either inside the cells or after cell lysis, collect clonal barcoded templates with targeted first strand cDNA and further prime the first strand cDNA with a second set of target specific primers to generate double stranded DNA and tagmentated with transpososomes, such as Mu and Tn5. The tagmentated double-stranded cDNA fragments can be used for downstream applications, such as, PCR assay and/or library construction for sequencing.

In one embodiment, barcoded microparticles with a set of target specific primers are used for in-cell capture of specific nuclei acid target(s). Transfect clonal barcode templates and target specific primers into the cells without compartmentation. Perform reverse transcription reaction either inside the cells or after cell lysis, collect clonal barcoded templates with targeted first strand cDNA. The RNA/DNA hybrid double-stranded molecule can be tagmentated with transpososomes such as Mu and Tn5. The tagmentated RNA/DNA hybrid double-stranded fragments can be used for downstream applications, such as, PCR assay and/or sequencing.

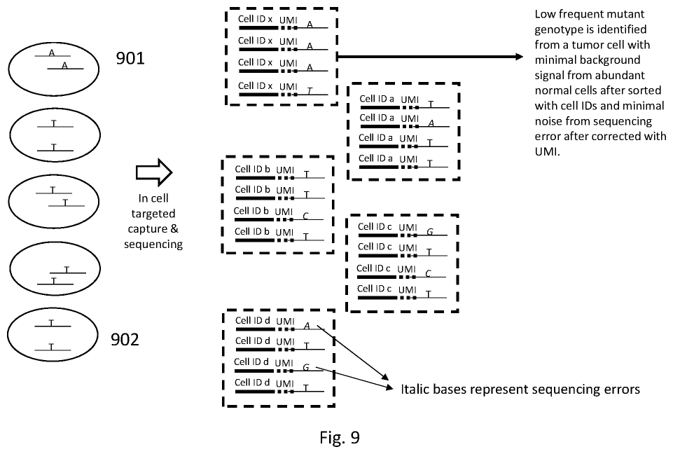

In-cell capture method described in this invention will make barcoding every single cell feasible operationally and economically. With the ability to uniquely barcode label all the cells or most cells, we can detect any mutations at a single cell level, which will effectively eliminate the background noise from surrounding cells. This will solve the sensitivity problem for detecting very low frequent somatic mutations which is required for early cancer detection. FIG. 9 illustrates the power of genotyping at a single cell level. There is a cell containing a mutant allele A (901), but there are many wild type cells containing a normal allele T (902) in the same sample. After in-cell capture with cell unique barcode, molecule unique UMI and sequencing, we can group sequencing reads based on their cell ID. For each cell, we can identify sequencing error based on UMI and make a correct variant calling easily. This approach can be applied for circulating tumor cells, tissue biopsy samples or tissue sections.

In-cell targeted capture can be used for identifying antibody heavy chain and light chain pairing, T cell alpha and beta chain pairing, and general immune repertoire profiling when applied for B cell and T cell samples.

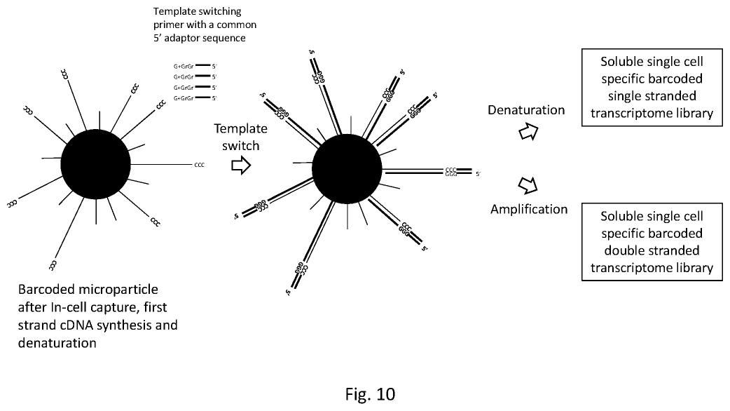

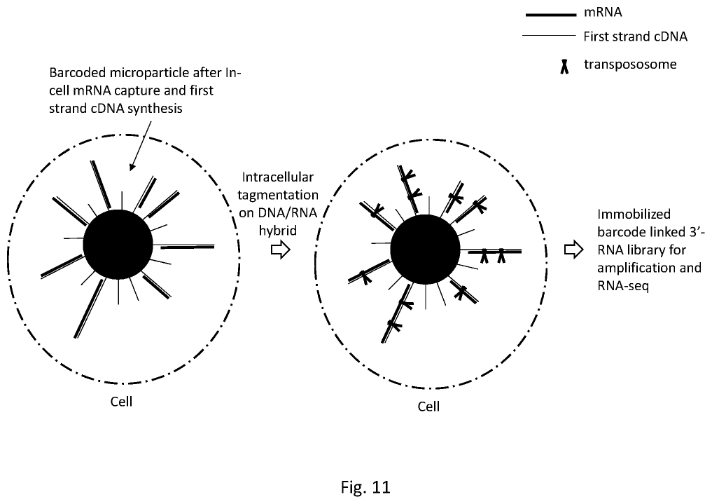

In-cell capture can also be used for single cell transcriptome profiling when poly-T tailed primer and/or random primers used as a capture sequence on the barcode template for in-cell capture. One embodiment is to use barcoded microparticles for in-cell capture of messenger RNA, first strand cDNA synthesis inside or outside the cells, and template switch reaction inside or outside the cells for whole transcriptome analysis (FIG. 10). Another embodiment is to use barcoded microparticle for in-cell capture of messenger RNA, reverse transcribe the mRNA inside or outside the cells and tagment the RNA/DNA hybrid double-stranded fragments using transpososomes, such as MuA, or Tn5 inside or outside the cells for whole transcriptome analysis (FIG. 11).

In-Cell Barcoded Capture of Protein

In one embodiment, a protein capture moiety is attached to a first barcoded template with a unique barcode sequence. Many different protein capture moieties are attached with barcode templates each with a different first barcode sequence. The protein capture moiety can be an antibody, antibody derivatives, affibody, nanobody, aptamer, or protein ligand. Transport one or more different protein capture moieties into the cells. Transfect a plurality of a second clonal barcode template into the cells without compartmentation, wherein the second barcode template can hybridize to the first barcode template on the protein capture moiety, which captures endogenous protein inside the cell. Break the cells and release the barcode attached endogenous protein.

Sequence the first and second barcode templates. Based on barcode quantitation and identity, we can measure the level of endogenous protein (first barcode) on a per cell basis (second barcode).

In some embodiment, other molecules, such as chemical compounds can also be the target of In-Cell capture when the first barcoded sequences are attached to these molecules.

In some embodiment, a second clonal barcode templates can be used to capture nucleic acid targets inside the cell at the same time of capture of endogenous protein targets.

Although the invention has been explained with respect to an embodiment, it is to be understood that many other possible modifications and variations can be made without departing from the spirit and scope of the invention as herein described.

Further, in general with regard to the processes, systems, methods, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments and should in no way be construed so as to limit the claimed invention.

Moreover, it is to be understood that the above description is intended to be illustrative and not restrictive. Many embodiments and applications other than the examples provided would be apparent to those of skill in the art upon reading the above description. The scope of the invention should be determined, not with reference to the above description, but should instead be determined with reference to the appended claims, along with the full scope of equivalents to which such claims are entitled. It is anticipated and intended that future developments will occur in the arts discussed herein, and that the disclosed systems and methods will be incorporated into such future embodiments. In sum, it should be understood that the invention is capable of modification and variation and is limited only by the following claims.

Lastly, all defined terms used in the application are intended to be given their broadest reasonable constructions consistent with the definitions provided herein. All undefined terms used in the claims are intended to be given their broadest reasonable constructions consistent with their ordinary meanings as understood by those skilled in the art unless an explicit indication to the contrary is made herein. In particular, use of the singular articles such as “a,” “the,” “said,” etc. should be read to recite one or more of the indicated elements unless a claim recites an explicit limitation to the contrary.

### EXAMPLES

### Example 1

Preparation of poly-T extended clonal barcoded beads

TELL beads, 3 μm clonally barcoded beads were from TELL-Seq WGS Library Prep Kit (UST Corporation, PN #100000). 3′ end poly-T extended TELL beads were prepared using Pfu DNA polymerase and a polyA-UMI (unique molecular identifier) oligo A22-tUMI10 (5′-NBAAAAAAAAAAAAAAAA/WAABNNNNNNNNNGTGACCTGTCCCAGCGTCTCCA C-3′ (SEQ ID NO: 1)) for a primer extension of TELL beads and described (FIG. 1) as following.

Eight 50 μL reactions of 1×Pfu buffer, 1 mM dNTP, 2.5 mM MgCl2, 0.5 μM A22-tUMI10, 20 million TELL beads, and 0.06 U/μL Pfu polymerase were prepared. The following PCR program was used: 95° C. for 1 minute, followed by 10 cycles of 95° C. for 10 seconds, 62° C. for 45 seconds, and 72° C. for 45 seconds, followed by 72° C. for 3 minutes. After PCR, all the beads were combined and washed three times with bead wash buffer (10 mM Tris HCl, 0.1 mM EDTA, 0.1% tween, pH 8). The beads were then stripped by resuspending the beads in 500 μL of freshly diluted 0.2 N NaOH and incubated for 5 minutes. The beads were then washed three times with 0.2 N NaOH to remove all stripped oligos and another three times with bead wash buffer was to remove all traces of NaOH. The beads were resuspended in bead wash buffer at a concentration of 500,000 beads/μL.

### Example 2

Transfection of Barcoded Beads into Cells for In-Cell Capture

HCT116 cells were cultured and maintained with DMEM media (Thermo Fisher Scientific, PN #11965-092) supplemented with 10% FBS (Thermo Fisher Scientific, PN #26140-079), 1× Penicillin/streptomycin (Thermo Fisher Scientific, PN #15140-122), 1× Glutamax (Thermo Fisher Scientific, PN #35050-061), and 0.05 mM 2-mercaptoethanol (Thermo Fisher Scientific, PN #21985-023). For RNA extraction, when cells reached ˜75% confluency (approximately 1 million cells), cells were lysed and RNA purified using Qiagen's RNeasy kit (Qiagen, PN #74104). The manufacturers protocol was followed with an on-column DNase treatment (Qiagen, PN #79254) and RNA purification step (required an additional DNase treatment). RNA was quantified using a Broad Range Qubit assay (Thermo Fisher Scientific, PN #Q10210).

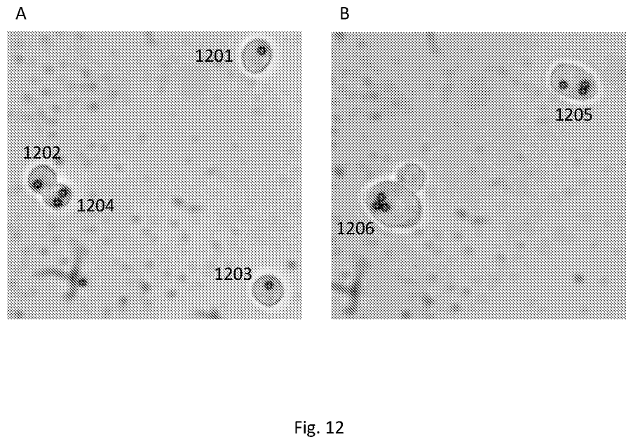

Once HCT116 cells reached about 80-90% confluency (approximately 1-1.5M cells), they were transfected with poly-T extended TELL beads. To prepare beads for transfection, 500 μL of complete DMEM media without FBS were added to each of four 1.5 mL protein low-bind microcentrifuge tubes. 2 μL of a previously prepared 10 ng/μL (w/v) PEI stock solution was added to each tube containing DMEM media. 3 μL of 500,000/μL poly-T extended TELL beads was added to each tube and immediately vortexed for one second each at max speed. The beads were incubated in the DMEM-PEI solution at room temperature for 30 minutes. The media on the cells was removed and the cells were washed twice with PBS to remove any residual FBS. PEI-coated beads from the four tubes were pooled then added to the cells. The plate of cells was placed on top of an OzBioscience plate magnet (OzBiosciences PN #MF10000) and then placed in a 37° C. 5% CO2 incubator for 3 minutes. The magnet was removed, and the cells were left in the incubator for 1 hour. After the incubation, the media was removed, and the cells washed 1× with PBS. 200 μL 0.125% trypsin was added to the cells and placed in the incubator for 3 minutes. 800 μL of DMEM media with 10% FBS was then added and the cells were mixed by pipetting 10 times. The cells were transferred to a 1.5 mL protein low-bind microcentrifuge tube. Using the OzBioscience magnet, the cells were placed against the edge of the magnet for 2 minutes. The transfected cells attached to the wall of the microcentrifuge tube while the non-transfected cells stay in solution. The negative cells were removed and placed in a new microcentrifuge tube. The positive cells and non-transfected beads were purified two more times by resuspending in 1 mL of hypotonic resuspension buffer (10 mM Tris-HCl pH 7.4, 10 mM NaCl, 3 mM MgCl₂) while removing negative cells from solution. The final resuspension was in a volume of 25 μL resuspension buffer. The positive cells and negative cells were then counted by hemocytometer. On average, 40% of cells were transfected with one bead. A transfection rate as high as 75% has been observed with the addition of more beads during transfection. For cells with bead transfected, some cells contained only one (1201, 1202 and 1203) or two 3 μm TELL beads (1204) in FIG. 12A; others contained more than two TELL beads (1205 and 1206) in FIG. 12B.

### Example 3

In Situ Reverse Transcription in Bead Transfected Live Cells

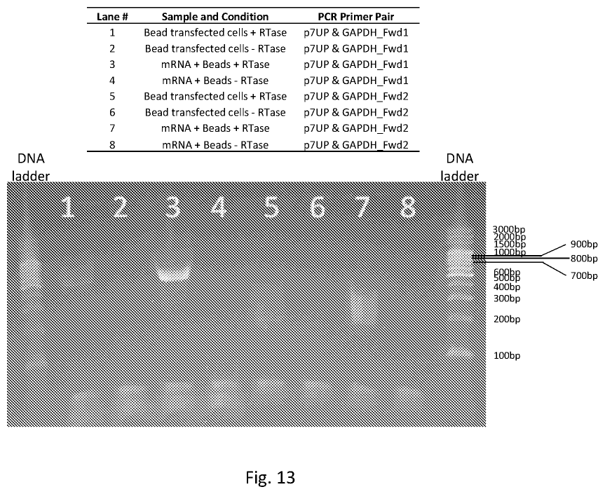

Superscript IV First-Strand Synthesis System kit (Thermo Fisher Scientific, PN #18091050), reverse transcription (RT) was performed on the live cells. The manufacturer's recommended protocol was performed using approximately 150,000 bead transfected cells from Example 2 as input. 500,000 poly-T extended TELL beads with 500 ng total RNA was used as a positive control and a no reverse transcriptase as a negative control. Final RNase H treatment described in the manufacturer's protocol was not performed. After reverse transcription, 200 μL of resuspension buffer was added to the RT mixture and purified by capturing the beads/cells on the magnet for 2 minutes. The solution was removed and only the cells/beads were left and attached to the side of the tube. A total of three washes were performed and the final beads/cells were resuspended in 25 μL resuspension buffer. To confirm the reverse transcription reaction, a PCR reaction was performed using 1× Phusion, 1 μL of reverse transcription product, and a TELL bead-specific primer, P7UP (5′-CAAGCAGAAGACGGCATACGAGATCCAGAGCCTCTCTATGGGCAG-3′ (SEQ ID NO: 2)) with a GAPDH-specific primer, GAPDH_Fwd1 primer (5′-CTGGGCTACACTGAGCACC-3′ (SEQ ID NO: 3)), which was approximately 400 bp away from the poly-A tail of GAPDH mRNA. This PCR should be able to amplify a ˜530 bp product (FIG. 13 Lane 1 and Lane 3) when GAPDH mRNA was captured by the poly-T extended TELL beads and reverse-transcribed to generate first strand cDNA using poly-T sequences on the beads as a RT primer. Lane 3 in FIG. 13 was a positive control for both capture of mRNA and RT reaction on the beads. Lane 1 in FIG. 1 was the result of successful In-Cell capture of GAPDH mRNA to poly-T extended TELL beads and in situ reverse transcription to generate first strand GAPDH cDNA. Also, another GAPDH-specific primer, GAPDH-Fwd2 (5′-GAGCCGCACCTTGTCATGTAC-3′ (SEQ ID NO: 4)) primer, which was 50 bp away from the poly-A tail GAPDH mRNA was used. This PCR product should be ˜180 bp (FIG. 13 Lane 5 and Lane 8) when GAPDH mRNA was captured by the poly-T extended TELL beads and reverse-transcribed to generate first strand cDNA using poly-T sequences on the beads as a RT primer. Similarly, Lane 7 in FIG. 13 was a positive control for both capture of mRNA and RT reaction on the beads. Lane 5 in FIG. 1 was the result of successful In-Cell capture of GAPDH mRNA to poly-T extended TELL beads and in situ reverse transcription to generate first strand GAPDH cDNA. PCR cycling conditions included 1 min at 98° C., followed by 24-28 cycles of 98° C. for 15 seconds, 60° C. for 15 seconds, 72° C. for 15 seconds followed by 1 cycle of 72° C. for 2 minutes. The PCR products appeared smear like bands on the agarose gel were due to reverse transcription started at different location of poly-A tail of the GAPDH mRNA.

### REFERENCES

Antimisiaris S G, et al. 2018. Pharmaceutics 10:218.
Bagnoli J W, et al. 2018. Nat Commun 9: 2937.
Bassell G J, Powers C M, Taneja K L, Singer R H. 1994. J Cell Biol 126:863-876.
Boussif O, et al. 1995. Proc Natl Acad Sci USA 92:7297-7301.
Braun T, et al. 2016. Cytometry A 89 (3): 301-308.
Datlinger P, et al. 2017. Nature Methods 14:297-301.
de Bruin K, et al. 2007. Mol Therapy 15:1297-1305.
Eloy J O, et al. 2017. Anticancer Agents Med Chem 17: 48-56.
Eloy J O, et al. 2017. Colloids Surfaces B Biointerfaces 159: 454-467.
Grün D, et al. 2015. Nature 525:251-255.
Hughes C, et al. 2001. Mol Therapy 3:623-630.
Huth S, et al. 2004. J Gene Med 6:923-936.
Janiszewska M, et al. 2015. Nature Genetics 47:1212-1219.
Kam Y, et al. 2012. Mol Pharm 9: 685-693.
Kawaguchi A, et al. 2008. Development 135:3113-3124.
Klein A M, et al. 2015. Cell 161:1187-1201.
Krotz F, et al. 2003. Mol Therapy 7(5):700-710.
Kurihara K, et al. 2011. Nat Chem 3: 775-781.
Laouini A, et al. 2012. J Colloid Sci Biotechnol 1: 147-168.
Li L, et al. 2017. Cell Stem Cell 20:858-873.
Lu J, et al. 2018. Immunol. Res 66:313-322.
Macosko E Z, et al. 2015. Cell 161:1202-1214.
McKnight T E, et al. 2004. Nano Letters 4(7):1213-1219.
Miranda D and Lovell J F. 2016. Bioeng Transl Med 1: 267-276.
Namgung R, et al. 2010. Biomaterials 31:4204-4213.
Okabe K, et al. 2011. Nucleic Acids Res 39: e20.
Parker A L, et al. 2003. Expert Rev Mol Med 5: 1-15.
Pazzi J, et al. 2018. Langmuir: doi:10.1021/acs.langmuir.8b03076 [Epub ahead of print].
Pereno V, et al. 2017. ACS Omega 2: 994-1002.
Picelli S, et al. 2013. Nature Methods 10:1096-1098.
Picelli S, et al. 2014. Nature Protocols 9:171-181.
Plank C, et al. 1994. J Biol Chem 269:12918-12924.
Raz-Ben Aroush D, et al. 2015. Methods in cell biology 125: 409-422.
Rodrigo J P, et al. 2005. Head Neck 27:995-1003.
Sato Y T, et al. 2016. Sci. Rep 6:21933.
Sauer A M, et al. 2009. J Control Release 137:136-145.
Scherer F, et al. 2002. Gene Therapy 9:102-109.
Shalek A K, et al. 2013. Nature 498:236-240.
Shapiro E., Biezuner T. and Linnarsson S. 2013. Nature Reviews Genetics 14:618-630.
Spitzer M H and Nolan G P. 2016. Cell 165:780-791.
Tang F, et al. 2009. Nature Methods 6:377-382.
Treutlein B, et al. 2014. Nature 509:371-375.
Uchida M, et al. 2009. BBA General Subject 1790(8):754-764
Zeisel A, et al. 2015. Science 347:1138-1142.
Zheng C, et al. 2017. Cell 169:1342-1356.
Zheng G X Y, et al. 2017. Nat Commun 8: 14049.

1. A method for intracellular barcoding targets without compartmentation comprising:
a. providing a barcode template;
b. providing a cell;
c. transfecting the barcode template into the cell; and wherein when the cell is in the context of a plurality of other cells, the cell is not separated by a partition from the other cells, and wherein the barcode template captures an intracellular target inside the cell;
d. generating a nucleic acid sequence derived from the captured intracellular target inside the cell or from the captured intracellular target after the cell is lysed, and wherein the nucleic acid sequence is attached to a barcode sequence or a complementary barcode sequence from the barcode template; and
e. identifying the nucleic acid sequence and/or its complementary sequence as being derived from the cell based on the presence of the barcode sequence attached; and wherein another nucleic acid sequence with the same barcode sequence correlates with the same cell.

2. The method of claim 1, wherein the barcode template is amongst a plurality of barcode templates that are immobilized on a microparticle; wherein the microparticle size ranges from about 100 nm to about 100 μm or from 1 μm to 20 μm; wherein optionally the microparticle is magnetic or degradable; and wherein the plurality of barcode templates are clonal with respect to each other.

3. (canceled)

4. (canceled)

5. The method of claim 2, wherein the microparticle is among a plurality of microparticles; wherein a population within the plurality of microparticles are non-barcoded and another population within the plurality of microparticles comprise barcode templates; wherein the two populations are mixed; and wherein the barcode templates in the population of microparticles with barcode templates are clonal with respect to each microparticle.

6. The method of claim 1, wherein the barcode template is non-immobilized.

7. The method of claim 6, wherein the non-immobilized barcode template is encapsulated in a liposome or a droplet; wherein within each liposome or each droplet, the barcode template is clonal with respect to another barcode template in the liposome or in the droplet.

8. The method of claim 1, wherein a clone of the barcode template comprises about 10 or more copies of the barcode template, or about 10,000 or more copies, or about 10,000,000 or more copies.

9. (canceled)

10. (canceled)

11. The method of claim 1, wherein on a microparticle or within a liposome or a droplet, the barcode template is clonal with respect to another barcode template in a population of barcode templates; and wherein more than one population of barcode templates exists on the microparticle or within the liposome or the droplet.

12. The method of claim 1, wherein on a microparticle or within a liposome or a droplet, more than one population of barcode templates exists, wherein the first population of barcode template is clonal with respect to the first population of another barcode template in a plurality of barcode templates; and the second population of barcode template is the same with respect to the second population of another barcode template in a plurality of barcode templates.

13. The method of claim 1, wherein the barcode template is among a plurality of barcode templates and the cell is among a plurality of cells; wherein a ratio of the barcode templates to the cells is such that when the barcode templates are transfected into the cells, less than about 30% of the transfected cells comprise more than one clonal population of barcode templates, and more than about 70% of the transfected cells comprise one or less clonal population of barcode templates; or wherein when the barcode templates are transfected into the cells, less than about 20% of the transfected cells comprise more than one clonal population of barcode templates, and more than about 80% of the transfected cells comprise one or less clonal population of barcode templates; or wherein when the barcode templates are transfected into the cells, less than about 10% of the transfected cells comprise more than one clonal population of barcode templates, and more than about 90% of the transfected cells comprise one or less clonal population of barcode templates.

14. (canceled)

15. (canceled)

16. The method of claim 1, wherein within the cell, more than one population of a plurality of barcode templates is in the cell; and wherein the barcode templates within each population of barcode templates are clonal.

17. The method of claim 1, wherein the barcode template compromising a barcode sequence, and at least one adaptor capable of priming, hybridization, amplification, strand transfer, or a combination thereof; wherein optionally the adaptor is selected from the group consisting of a ploy-T sequence, a target specific sequence, a pool of different target specific sequence, a random degenerate sequence, and a combination thereof.

18. The method of claim 1, wherein one clone of the barcode template comprising an UMI sequence.

19. (canceled)

20. The method of claim 1, wherein the intracellular target is selected from the group consisting of a RNA, a DNA, an oligonucleotide, an oligonucleotide-labeled protein, an oligonucleotide-labeled chemical compound, and a combination thereof; wherein optionally the intracellular target enters the cell as an extracellular component through a specific recognition of the cell.

21. (canceled)

22. The method of claim 1, wherein the cell is selected from the group consisting of a cultured cell, a blood cell, a tissue, a tissue section, a biopsy sample, a nucleus derived from the cell, and a combination thereof.

23. The method of claim 1, wherein the cell is fixed with a fixative selected from the group consisting an alcohol, a Hepes-glutamic acid buffer-mediated organic solvent protection effect (HOPE) fixative, and a combination of thereof.

24. (canceled)

25. The method of claim 1, wherein the target capture is mediated by hybridization, ligation, strand transfer, directly or indirectly, or a combination thereof.

26. The method of claim 1, wherein the generated nucleic acid sequence derived from the captured intracellular targets is by reverse transcription, primer extension, ligation, amplification, tagmentation, or a combination thereof.

27. The method of claim 1, wherein the intracellular barcoded target is used for single cell expression profiling, single cell targeted sequencing, immune repertoire analysis and/or single cell protein analysis.

28. (canceled)

29. (canceled)

30. A method for cell-specific intracellular nucleic acid barcoding without compartmentation comprising:
a. providing a barcode template, wherein the barcode template comprises a cell-specific anchor;
b. providing a cell;
c. contacting the clonal barcode template of (a) and the cell of (b), and anchoring a clone of the barcode template to a specific cell type by the cell-specific anchor;
d. transfecting the clonal barcode template into the cell wherein the cell is not compartmentalize with respect to another cell, and wherein the barcode template hybridizes to a nucleic acid target inside the cell;
e. generating one or more nucleic acid sequences derived from the barcode template and the hybridized nucleic acid target inside the cell or after cell is lysed, wherein the one or more nucleic acid sequences are attached to the same barcode sequence from the barcode template in the cell; and
f. sequencing the nucleic acid sequence to characterize the nucleic acid target in the specific cells on a per cell basis based on the presence of a common barcode sequence.

31. (canceled)

32. A method for early disease detection comprising:
a. providing a test sample comprising a cell or cellular component;
b. barcoding a nucleic acid inside the cell or cellular component to generate a cellular barcode-tagged nucleic acid sequence;
c. generating a sequencing library wherein the sequencing library comprises a region containing one or more disease-causative variants and the cellular barcode tag using the barcode-tagged nucleic acid sequences in (b);
d. grouping sequencing read based on their cellular barcode sequences and determining the presence of disease-causative variants on a per cell basis; and
e. counting the number of the cells containing disease-causative variants among the test sample.

33. (canceled)
